(19) United States
(12) Patent Application Publication
(10) Pub. No.: US 2023/0062280 A1
(43) Pub. Date: Mar. 2, 2023

(54) SYSTEMS AND METHODS FOR CEREBRAL IMPLANTATION STRATEGIES FOR DELIVERY OF ALTERNATING ELECTRIC FIELD THERAPY

(71) Applicant: Medtronic, Inc., Minneapolis, MN (US)

(72) Inventor: Benjamin Kevin Hendricks, Phoenix, AZ (US)

(21) Appl. No.: 17/929,595

(22) Filed: Sep. 2, 2022

Related U.S. Application Data
(60) Provisional application No. 63/240,243, filed on Sep. 2, 2021.

(51) Int. Cl. A61B 34/10; A61N 1/40; A61B 34/00; G06T 17/20

(52) U.S. Cl. CPC A61B 34/10; A61N 1/40; A61B 34/25; G06T 17/20; A61B 2034/104; A61B 2034/105

(57) ABSTRACT

Various embodiments for system and method for cerebral implantation strategy for delivery of alternating electric field therapy are described. For example, a system may include processing circuitry configured for operative communication with a conformable grid comprising a plurality of modular grid elements having a plurality of electrodes configured for implantation in a cerebrum, and wherein the processing circuitry is configured to execute instructions stored in the memory to model brain tissue to define inter-contact and intra-contact distances along the conformable grid and each of the plurality of electrodes; determine the spacing between the plurality of modular grid elements of the conformable grid. A user interface may display a visual representation of the cerebrum including identification of a sub-region of the cerebrum and display a representation of the spacing between the plurality of modular grid elements of the conformable grid and a depth of each electrode of the plurality of electrodes.

This application claims the benefit of U.S. Provisional Patent Application No. 63/240,243, filed Sep. 2, 2021, the entire contents of which is incorporated herein by reference.

### TECHNICAL FIELD

The present disclosure generally relates to the treatment of cancer, and in particular to techniques for cerebral device implantation and the treatment of brain cancer via electric field generation.

### BACKGROUND

Glioblastoma (GBM) is the most common primary brain malignancy, defined as a grade IV astrocytic lesion by the World Health Organization. Current standard of care has made marginal advances in median survival approximating 15.6 months. Traditional standard of care for patients diagnosed with GBM includes maximal safe surgical resection, adjuvant temozolomide, and adjuvant radiotherapy. One recent addition to the Food and Drug Administration (FDA) approved standard of care has been Tumor-treating fields (TTF), otherwise referred to in this manuscript as alternating electric fields (AEF) delivered by the Optune device (Novocure Ltd.), which in 2015 demonstrated a significant increase in progression-free survival and overall survival in a randomized controlled trial of TTF+temozolomide versus temozolomide alone (7.1 vs. 4.0 months, P=0.001 and 20.5 versus 15.6 months, P=0.004, respectively). TTF were delivered as a 200-kHz AEF generated by 4 cutaneous transducer arrays applied to the scalp and connected to a portable handheld battery pack for >18 hours/day.

### SUMMARY

Techniques, systems, and devices configured to deliver AEF therapy to the brain are described. In one example, a system can be configured to anatomically divide a brain and appropriate regional electrode configurations selected to permit appropriate placement of implantable electrodes for AEF therapy to a particular target. The system can be configured to determine one or more parameters that define the AEF and enable the delivered AEF to impact cellular physiology, such as the inhibition of tumor cell division.

In one example, a system includes processing circuitry configured for operative communication with a conformable grid, a user interface, and a memory, wherein the conformable grid comprises a plurality of modular grid elements having a plurality of electrodes configured for implantation in a cerebrum of a patient, and wherein the processing circuitry is configured to execute instructions stored in the memory to: model brain tissue to define inter-contact and intra-contact distances along the conformable grid and each of the plurality of electrodes; determine the spacing between the plurality of modular grid elements of the conformable grid; control the user interface to display a visual representation of the cerebrum including identification of a sub-region of the cerebrum; and control the user interface to display a representation of the spacing between the plurality of modular grid elements of the conformable grid and a depth of each electrode of the plurality of electrodes in the cerebrum within the sub-region of the cerebrum.

In another example, a method includes modeling, by processing circuitry, brain tissue to define inter-contact and intra-contact distances along a conformable grid and each of a plurality of electrodes, wherein the comfortable grid comprises a plurality of modular grid elements having the plurality of electrodes configured for implantation in a cerebrum of a patient; determining, by the processing circuitry, the spacing between the plurality of modular grid elements of the conformable grid; controlling, by the processing circuitry, a user interface to display a visual representation of the cerebrum including identification of a sub-region of the cerebrum; and controlling, by the processing circuitry, the user interface to display a representation of the spacing between the plurality of modular grid elements of the conformable grid and a depth of each electrode of the plurality of electrodes in the cerebrum within the sub-region of the cerebrum.

In another example, a computer-readable storage medium comprising instructions that, when executed, cause processing circuitry to model brain tissue to define inter-contact and intra-contact distances along a conformable grid and each of a plurality of electrodes, wherein the conformable grid comprises a plurality of modular grid elements having the plurality of electrodes configured for implantation in a cerebrum of a patient; determine the spacing between the plurality of modular grid elements of the conformable grid; control a user interface to display a visual representation of the cerebrum including identification of a sub-region of the cerebrum; and control the user interface to display a representation of the spacing between the plurality of modular grid elements of the conformable grid and a depth of each electrode of the plurality of electrodes in the cerebrum within the sub-region of the cerebrum.

### BRIEF DESCRIPTION OF DRAWINGS

FIG. 1 is an illustration showing an example segmented cerebral model based on a publicly available DICOM image set.

FIG. 2 is an image of the brain that highlights the lateral frontal sub-region within the frontal region of the cerebrum for illustrating placement of a conformable grid with projecting electrodes.

FIG. 3A is a perspective view of an example modular grid element of a conformable grid with projecting electrodes used in the cerebral implantation strategy; FIG. 3B is a top plan view of the modular grid element; FIG. 3C is a bottom plan view of the singular modular element; FIG. 3D is a left side view of the modular grid element; and FIG. 3E is a right side view of the modular grid element.

FIG. 4 is an illustration of an example depth electrode used in the cerebral implantation strategy.

FIG. 5 is a table showing example human brain tissue parameters obtained using the cerebral implantation strategy.

FIG. 6 is an image of the brain that highlights the media frontal sub-region within the frontal region of the cerebrum for illustrating placement of a conformable grid with projecting electrodes.

FIG. 7 is an image of the brain that highlights the basal frontal sub-region within the frontal region of the cerebrum for illustrating placement of a conformable grid with projecting electrodes.

FIG. 8 is an image of the brain that highlights the bilateral frontal sub-region within the frontal region of the cerebrum for illustrating placement of a conformable grid with projecting electrodes.

FIG. 9 is an image of the brain that highlights the lateral frontal sub-region within the temporal region of the cerebrum for illustrating placement of a conformable grid with projecting electrodes.

FIG. 10 is an image of the brain that highlights the medial temporal sub-region within the temporal region of the cerebrum for illustrating placement of a conformable grid with projecting electrodes.

FIG. 11 is an image of the brain that highlights the lateral parietal sub-region within the parietal region of the cerebrum for illustrating placement of a conformable grid with projecting electrodes.

FIG. 12 is an image of the brain that highlights the medial parietal sub-region within the parietal region of the cerebrum for illustrating placement of a conformable grid with projecting electrodes.

FIG. 13 is an image of the brain that highlights bilateral medial parietal sub-regions within the respective parietal regions of the hemispheres of the cerebrum for illustrating placement of a conformable grid with projecting electrodes.

FIG. 14 is an image of the brain that highlights the lateral occipital sub-region within the occipital region of the cerebrum for illustrating placement of a conformable grid with projecting electrodes.

FIG. 15 is an image of the brain that highlights the medial occipital sub-region within the occipital region of the cerebrum for illustrating placement of a conformable grid with projecting electrodes.

FIG. 16 is an image of the brain that highlights the basal occipital sub-region within the deep region of the cerebrum for illustrating placement of a conformable grid with projecting electrodes.

FIG. 17 is an image of the brain that highlights the insular sub-region within the deep region of the cerebrum for illustrating placement of a conformable grid with projecting electrodes.

FIG. 18 is an image of the brain that highlights the basal ganglia sub-region of the cerebrum for illustrating placement of a conformable grid with projecting electrodes.

FIG. 19 is a flow chart illustrating example aspects of cerebral implantation strategy.

FIG. 20 is an illustration of an example computer system configured to operate the cerebrum implantation strategy.

Corresponding reference characters indicate corresponding elements among the view of the drawings. The headings used in the figures do not limit the scope of the claims.

### DETAILED DESCRIPTION

This disclosure describes various devices, systems, and techniques for planning and/or delivering AEF therapy. As discussed above, external TTF delivery via the Optune device has been used to treat GBM. While the Optune device represents a great advancement in the treatment of GBM, there are aspects of AEF delivery that can be improved. A theoretical implantable delivery system for AEF therapy to a patient diagnosed with GBM would have numerous benefits over the transcutaneous system. The implantable system will require strategic lead placement to accomplish therapeutic delivery of sufficient electric field to the region of tumor, or high-risk region for tumor progression/recurrence.

As described herein, techniques and systems can be configured such that the brain can be anatomically divided and appropriate regional electrode configurations selected to permit optimal placement of implantable electrodes for alternating electric field (AEF) therapy to a particular target. The premise behind AEF therapy is that through delivery of AEF, cellular physiology is impacted in a potentially favorable, ex. selective tumor cell inhibition. This favorable cellular outcome is imparted by parameters of the AEF that are permissive for this cellular behavior, i.e. AEF frequency (kHz) and strength (V/cm). This method can also be applied to educate further device design within the realm of depth or grid electrode parameters (i.e. inter- and intra-contact distances).

AEF therapy has been described for use in a multitude of tumor/cancer types within the literature. This particular methodology for systematic organ evaluation could be applied to the treatment of a primary brain tumor, secondary (metastatic) brain tumor, or the prevention (i.e. prophylaxis) of primary or secondary brain tumors within the cranial vault; however, it can also be applied to systematic evaluation and lead design exploration in other organ systems.

This methodology in and of itself does not capacitate a product. It informs a proposed technology on the effective means by which AEF therapy can be delivered to brain tissue, and can be generalized to other organs and types of therapies.

It is with these observations in mind, among others, that various aspects of the present disclosure were conceived and developed.

Various embodiments for a system and method for a cerebral implantation strategy for implanting projecting electrodes relative to the cerebrum of a patient when conducting alternating electric field therapy for the treatment of cancer are disclosed herein. In one aspect, processing circuitry is in operative communication with a conformable electrode grid comprising a plurality of modular grid elements having a plurality of projecting electrodes configured for implantation within the cerebrum. In another aspect, the processing circuitry executes an application that provides a demonstration of a particular sub-region within the frontal, temporal, parietal, occipital and deep regions of the cerebrum that shows the spacing and depth of the electrodes during implantation of the electrodes of the conformable grid.

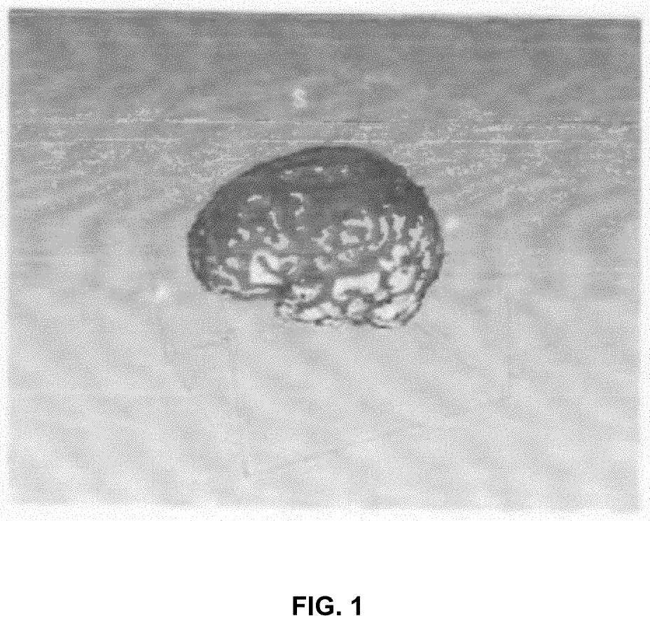

FIG. 1 shows a segmented cerebral model generated based on a publicly available DICOM image set. The system, such as system 100 described herein, may be configured to generate this segmented cerebral model. The example segmented cerebral model represents a segmented 3D mesh that separates the cerebrospinal fluid, white matter, and gray matter to permit finite element modeling (FEM) within 3-dimensional space which applies dielectric properties to these tissue types. This 3D mesh can then be manipulated by the system in a FEM environment with various electrodes to elucidate the adequate number and precise location of each electrode and contact based on a target zone. In this manner, the system may model brain tissue to define inter-contact and intra-contact distances along the conformable grid and each of the plurality of electrodes. The system may also determine, based on inter-contact and intra-contact distances along a confirmable grid and each of the plurality of electrodes of the grid and based on spacing between modular grid elements of the confirmable grid, the number of electrodes of a conformable grid for implantation and/or the location of implantation for the conformable grid and/or each of the electrodes of the conformable grid.

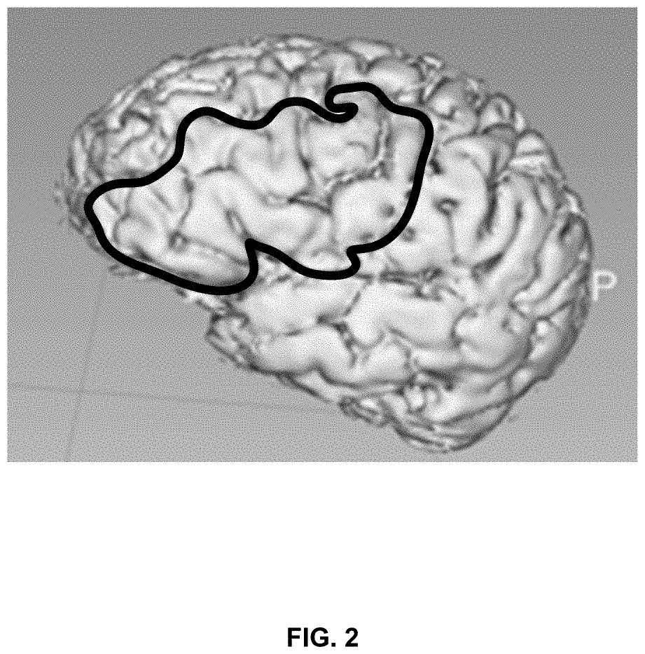

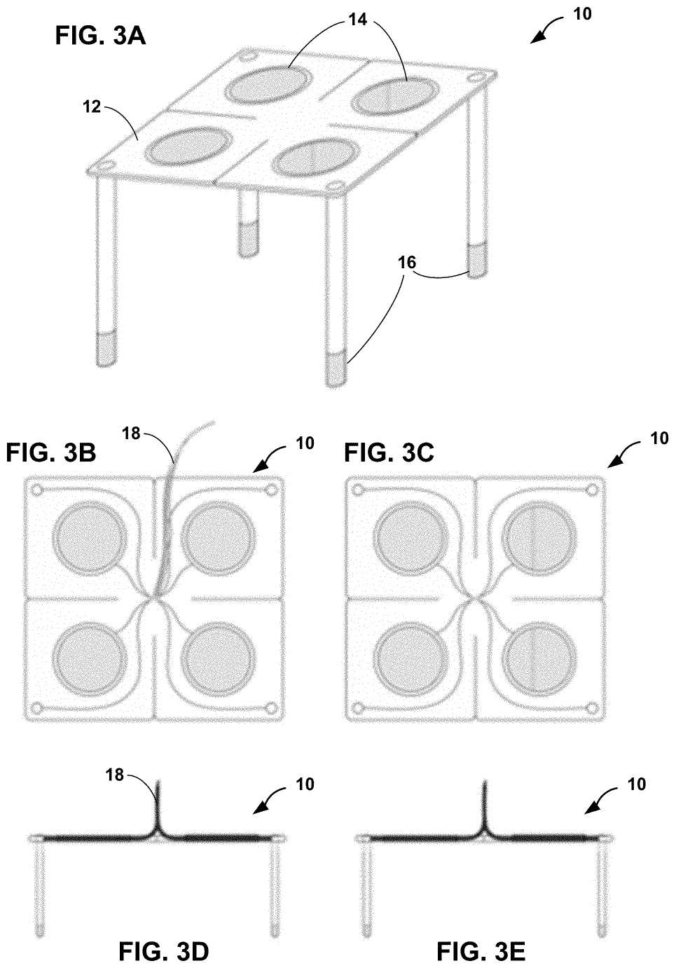

FIG. 2 is a demonstration of the “lateral frontal” sub-region within the “frontal” region of the cerebrum. This zone of cortical anatomy inclusive of the middle frontal gyrus, inferior frontal gyms, and caudal precental gyrus is projected to require an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3E) will be determined based on the FEM simulations. Depth electrodes may be necessary or desirable for supplemental application of AEF dependent on the FEM simulation results and are projected to be ideally positioned medially and posteriorly, given these are regions of anatomical subcortical connectivity within the brain housing the greatest volume of tumor tissue. The posterior positioning of electrodes must preserve integrity of the pre-central gyrus and associated corticospinal tract given the potential for adverse clinical outcomes.

FIGS. 3A-3E show various views of an example single modular element 10 of an example conformable grid with projecting electrodes 16, the specifications of which can be dependent on, or selected according to, the FEM experiments in a tissue dependent manner. FIG. 3A is a perspective view of an example modular grid element 10 of a conformable grid that can include two or more modular grid elements 10. Modular grid element 10 includes a structure 12 which can carry surface electrodes 14 and depth (or projecting) electrodes 16 carried by posts of modular grid element 10. Although modular grid element 10 includes four surface electrodes 14 and four depth electrodes 16, different number of surface and depth electrodes (e.g., as few as one or more than four) may be provided in another modular grid element 10. Modular grid element 10 may include the same number of surface and depth electrodes, or there may be a different number of depth electrodes to surface electrodes. Although one depth electrode is shown at the distal end of each of the posts of modular grid element 10, two or more depth electrodes may be provide on one or more (or all) of the posts at different positions along the length of the respective post.

FIG. 3B is a top plan view of modular grid element 10 and conductors 18 which includes a conductor for each respective electrode of modular grid element 10. FIG. 3C is a bottom plan view of the singular modular element 10. FIG. 3D is a left side view of the modular grid element 10, and FIG. 3E is a right side view of the modular grid element 10.

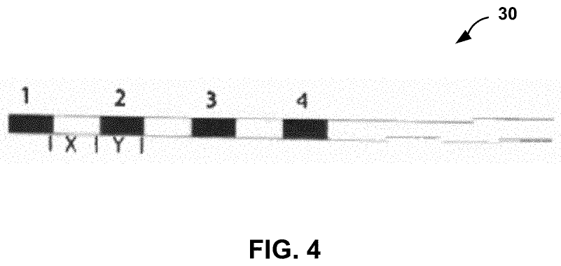

FIG. 4 is an illustration of a given depth electrode 30 configured to project into biological tissue and can be described by an inter-contact (X) and intra-contact (Y) distance which determines the spread of voltage into the adjacent tissue. Four contacts (i.e., individual electrodes) are shown for depth electrode 30, but fewer or greater numbers of electrodes may be provided in other examples. Depth electrode 30 may be an example of one depth electrode post on a modular grid element, such as modular grid element 10. FEM simulation within various organs permits an identification of an optimal intra-contact distance for the delivery of voltage to increase or maximize the local AEF field strength adjacent to the electrode versus at a farther distance from the electrode. Similarly, inter-contact distance evaluation in FEM simulation will permit an assessment of the target or ideal spacing of contacts that enables therapeutic AEF field strength between individual contacts of a given electrode.

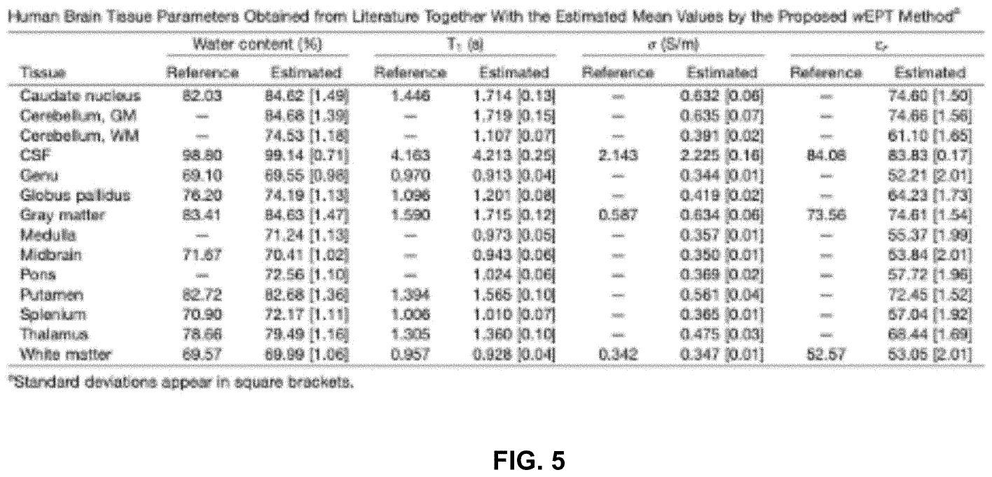

FIG. 5 is a table of human brain tissue parameters obtained from literature together with estimated mean values by the present method. Dielectric properties are variables within the in vivo cerebral anatomy that determine the distribution of AEF and therefore require FEM to attain accurate representations of the necessary implant strategies for clinical application. The brain tissue parameters have been Adopted from Michael E. et al. Electrical Conductivity and Permittivity Maps of the Brain Tissue Derived from Water Content Based on T1-Weighted Acquisition. Magnetic Resonance in Medicine 2017. 77:1094-1103. A system may generate the segmented cerebral model of the patient using these generalized parameters.

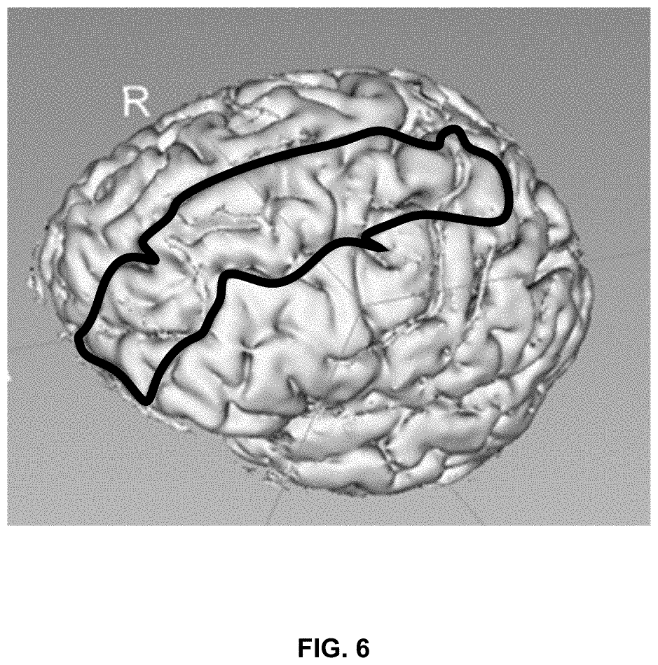

FIG. 6 is a demonstration of the “medial frontal” sub-region within the “frontal” region of the cerebrum. This zone of cortical anatomy inclusive of the superior frontal gyms, rostral precentral gyms, paracentral gyms, and cingulate gyrus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned lateral and posteriorly, given these are regions of anatomical subcortical connectivity within the brain housing the greatest volume of tissue at risk for tumor progression. Depth electrode entry will be within the middle or superior frontal gyms, if preserved following resection. The posterior positioning of electrodes must preserve integrity of the pre-central gyms and associated corticospinal tract given the potential for adverse clinical outcomes.

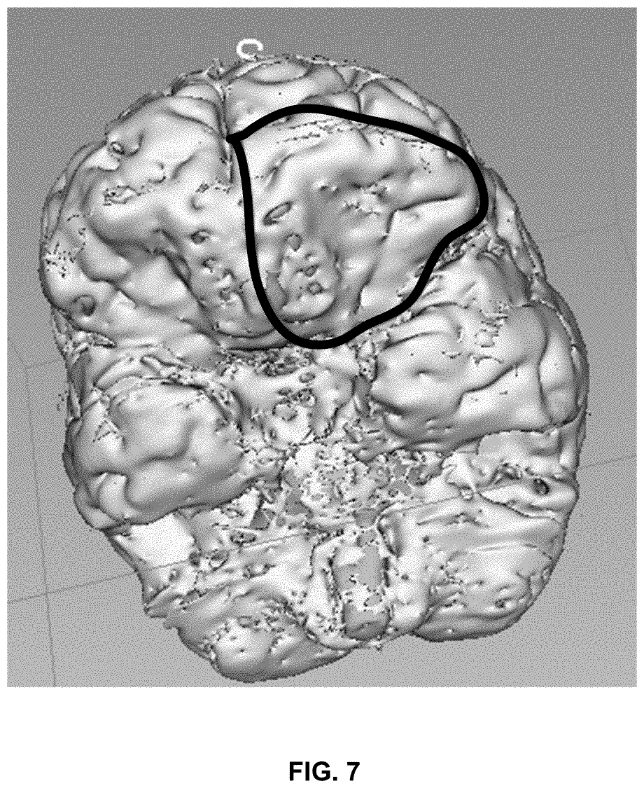

FIG. 7 is a demonstration of the “basal frontal” sub-region within the “frontal” region of the cerebrum. This zone of cortical anatomy inclusive of the gyrus rectus, orbital gyri (medial, lateral, anterior, and posterior), paraolfactory gyrus, and paraterminal gyrus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned superiorly and posteriorly, given these are regions of anatomical subcortical connectivity within the brain housing the greatest volume of tissue at risk for tumor progression. Depth electrode entry will be within the middle or superior frontal gyrus, if preserved following resection. The posterior positioning of electrodes must preserve integrity of the pre-central gyms and associated corticospinal tract, as well as avoiding the caudal margin of the inferior frontal gyms given the potential for adverse clinical outcomes related to Broca's area.

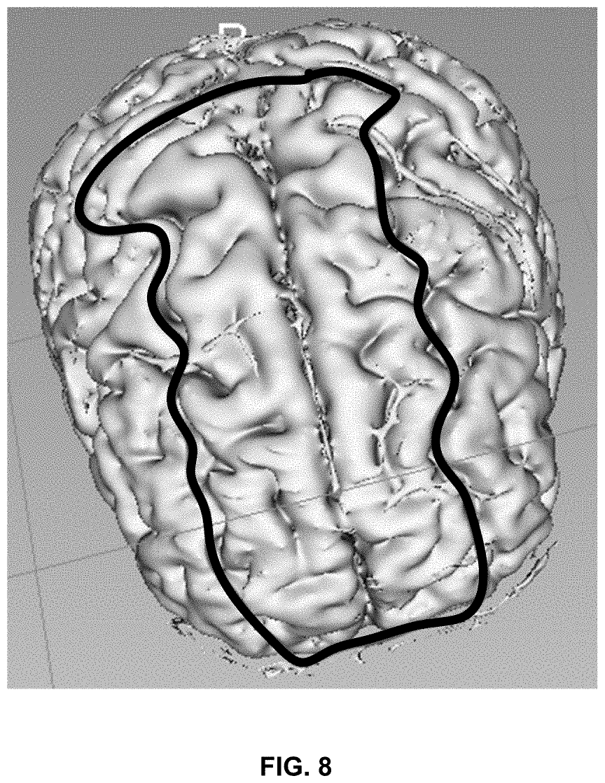

FIG. 8 is a demonstration of the “bilateral frontal” sub-region within the “frontal” region of the cerebrum. This zone of cortical anatomy inclusive of the superior frontal gyrus, rostral precentral gyms, paracentral gyms, cingulate gyms, corpus callosum—body, corpus callosum—genu, and corpus callosum—rostrum is projected to be optimally treated using an implantation strategy involving a resection cavity conformable grid with projecting electrodes (potentially multiple to accommodate bihemispheric implantation). The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned lateral and posteriorly, given these are regions of anatomical subcortical connectivity within the brain housing the greatest volume of tissue at risk for tumor progression. Depth electrode entry will be within the middle or superior frontal gyrus, if preserved following resection. The posterior positioning of electrodes must preserve integrity of the pre-central gyms and associated corticospinal tract given the potential for adverse clinical outcomes.

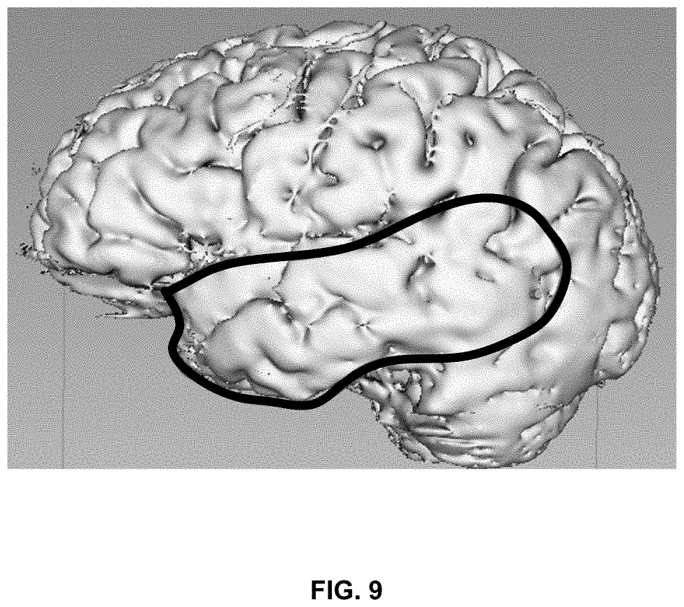

FIG. 9 is a demonstration of the “lateral temporal” sub-region within the “temporal” region of the cerebrum. This zone of cortical anatomy inclusive of the superior temporal gyrus, middle temporal gyms, and inferior temporal gyms is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the temporal stem and posteriorly within the temporal lobe, given these are regions of anatomical subcortical connectivity within the brain housing the greatest volume of tissue at risk for tumor progression. Depth electrode entry will be within the superior, middle, and inferior temporal gyrus, if preserved following resection. The posterior positioning of electrodes must avoid entry into Wernicke's area along the transverse temporal gyrus/posterior superior temporal gyrus given the potential for adverse clinical outcomes.

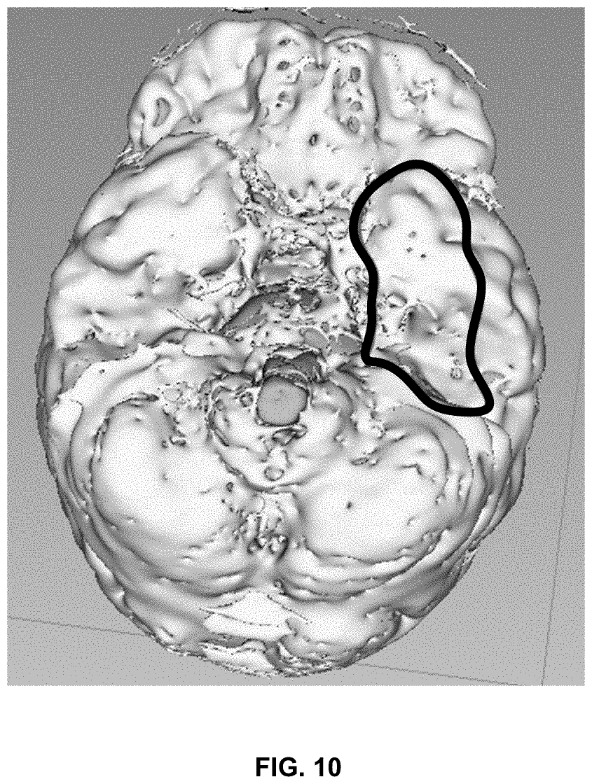

FIG. 10 is a demonstration of the “medial temporal” sub-region within the “temporal” region of the cerebrum. This zone of cortical anatomy inclusive of the occipitotemporal/fusiform gyms, parahippocampal gyrus, hippocampus, and uncus (gyms intralimbicus, uncinate gyms, limbus Giacomini) is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the temporal stem and posteriorly within the temporal lobe, given these are regions of anatomical subcortical connectivity within the brain housing the greatest volume of tissue at risk for tumor progression. Depth electrode entry will be within the middle and/or inferior temporal gyrus, if preserved following resection.

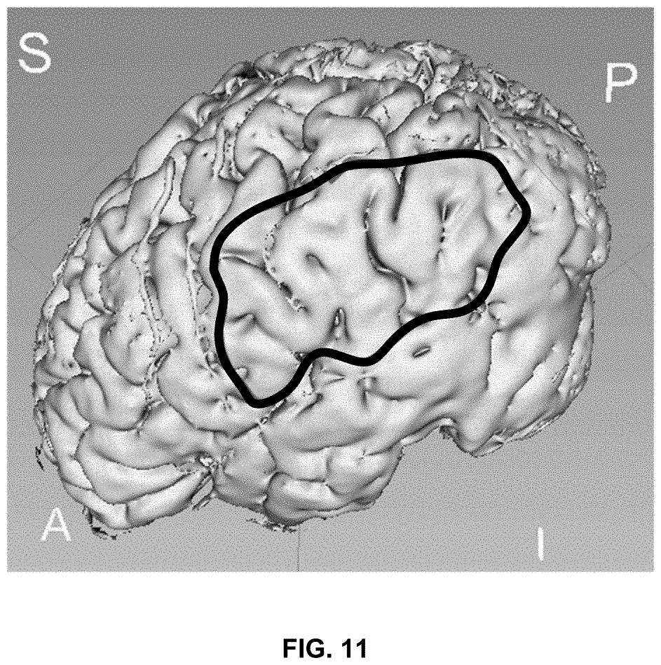

FIG. 11 is a demonstration of the “lateral parietal” sub-region within the “parietal” region of the cerebrum. This zone of cortical anatomy inclusive of the supramarginal gyms, angular gyrus, superior parietal lobule, inferior parietal lobule, and postcentral gyms is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the medial, posterior, and inferior margin, given these are regions of anatomical subcortical connectivity within the brain housing the greatest volume of tissue at risk for tumor progression. Depth electrode entry will be within the superior and/or inferior parietal lobule, if preserved following resection. Notably, implantation anteriorly should be cautiously performed within the post-central gyrus given the eloquence of this region.

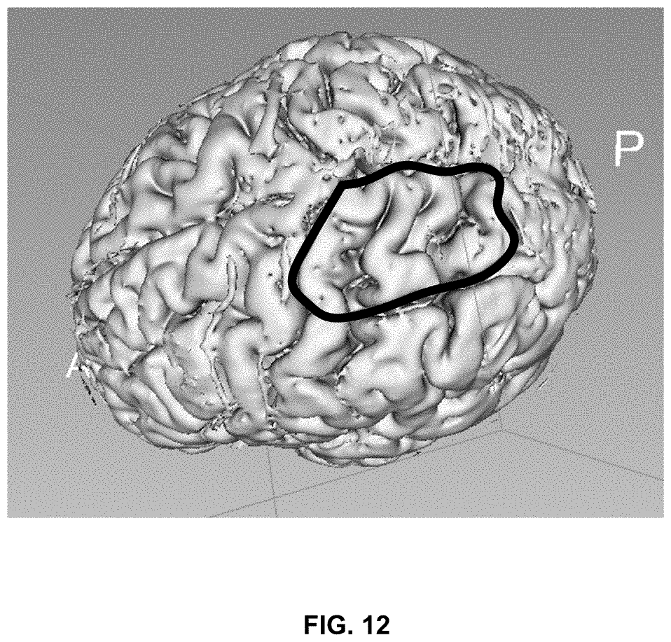

FIG. 12 is a demonstration of the “medial parietal” sub-region within the “parietal” region of the cerebrum. This zone of cortical anatomy inclusive of the posterior paracentral gyms, postcentral gyrus, superior parietal lobule, inferior parietal lobule, and precuneus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the lateral, posterior, and inferior margin, given these are regions of anatomical subcortical connectivity within the brain housing the greatest volume of tissue at risk for tumor progression. Depth electrode entry will be within the superior and/or inferior parietal lobule, if preserved following resection. Notably, implantation anteriorly should be cautiously performed within the post-central gyrus given the eloquence of this region.

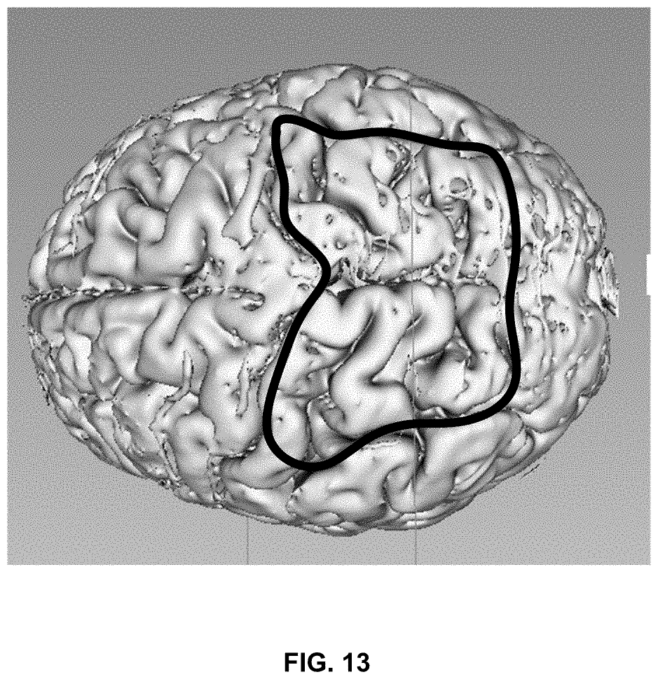

FIG. 13 is an image of the brain that highlights bilateral medial parietal sub-regions within the respective parietal regions of the hemispheres of the cerebrum for illustrating placement of a conformable grid with projecting electrodes. Similar to the discussion above with respect to FIG. 12, the zone of cortical anatomy inclusive of the posterior paracentral gyrus, postcentral gyrus, superior parietal lobule, inferior parietal lobule, and precuneus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the lateral, posterior, and inferior margin, given these are regions of anatomical subcortical connectivity within the brain housing the greatest volume of tissue at risk for tumor progression. Depth electrode entry will be within the superior and/or inferior parietal lobule of both hemispheres in this bilateral example, if preserved following resection. Notably, implantation anteriorly should be cautiously performed within the post-central gyrus given the eloquence of this region.

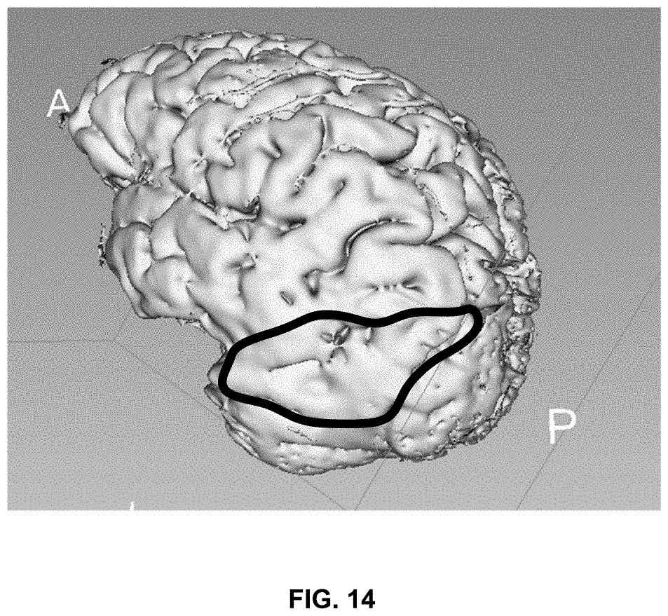

FIG. 14 is a demonstration of the “lateral occipital” sub-region within the “occipital” region of the cerebrum. This zone of cortical anatomy inclusive of the lateral portion of the superior occipital and inferior occipital gyrus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the anterior, and medial margin, given these are regions of anatomical subcortical connectivity within the brain housing the greatest volume of tissue at risk for tumor progression. Depth electrode entry will be within the superior and/or inferior occipital gyms, if preserved following resection. This region should be tolerant to depth electrode penetration and therefore the eloquence of the occipital region should not be a concern.

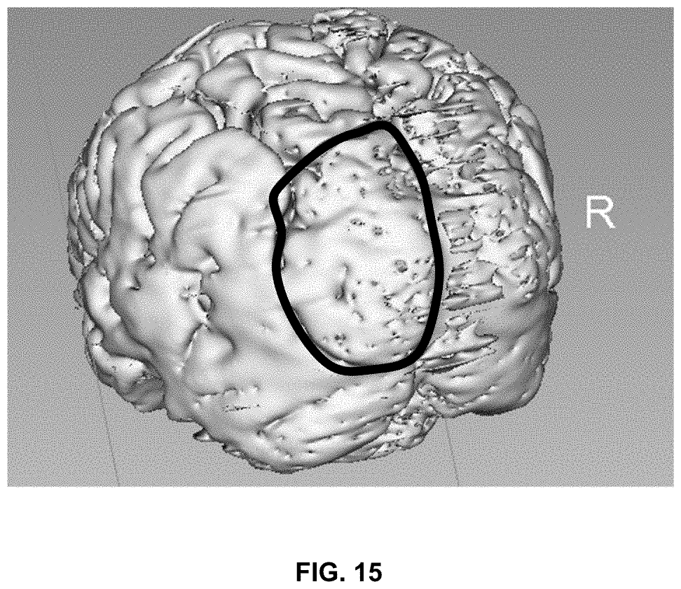

FIG. 15 is a demonstration of the “medial occipital” sub-region within the “occipital” region of the cerebrum. This zone of cortical anatomy inclusive of the medial portion of the superior occipital and inferior occipital gyrus is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the anterior and lateral margin, given these are regions of anatomical subcortical connectivity within the brain housing the greatest volume of tissue at risk for tumor progression. Depth electrode entry will be within the superior and/or inferior occipital gyms, if preserved following resection. This region should be tolerant to depth electrode penetration and therefore the eloquence of the occipital region should not be a concern.

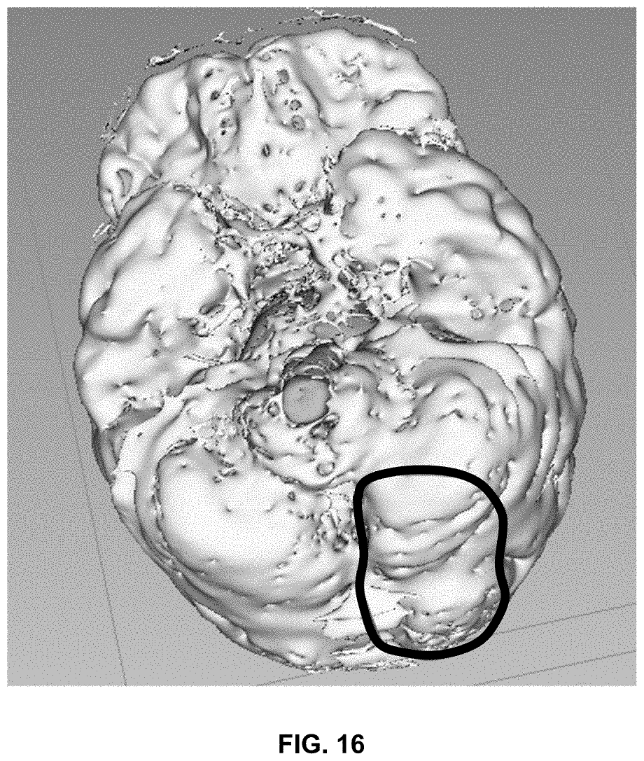

FIG. 16 is a demonstration of the “basal occipital” sub-region within the “deep” region of the cerebrum. This zone of cortical anatomy inclusive of the basal portion of the inferior occipital and lingula region of the occipital lobe is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3C) will be determined based on the FEM simulations. Depth electrodes may be necessary or desirable for supplemental application of AEF dependent on the FEM simulation results and are projected to be ideally positioned within the anterior and superior margin, given these are regions of anatomical subcortical connectivity within the brain housing the greatest volume of tissue at risk for tumor progression. Depth electrode entry will be within the superior occipital gyrus, if preserved following resection. This region should be tolerant to depth electrode penetration and therefore the eloquence of the occipital region should not be a concern.

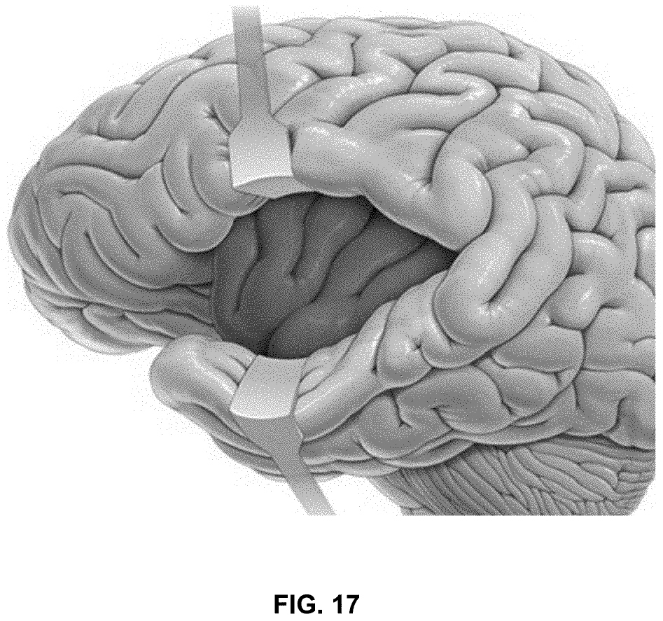

FIG. 17 is a demonstration of the “insular” sub-region within the “deep” region cerebrum. This zone of cortical anatomy inclusive of the basal portion of the short and long insular gyri and is projected to be optimally treated using an implantation strategy involving a resection cavity conformable grid with projecting electrodes. The inter- and intra-contact distances along the grid and projecting electrodes will be determined by the FEM simulation within the segmented model. Similarly, the spacing between the modular grid elements (described in FIGS. 3A-3E) will be determined based on the FEM simulations. Depth electrodes may be necessary or desirable for supplemental application of AEF dependent on the FEM simulation results and are projected to be ideally positioned within the anterior, media, and/or posterior margin, given these are regions of anatomical subcortical connectivity within the brain housing the greatest volume of tissue at risk for tumor progression. Depth electrode entry will be within the inferior or middle frontal gyrus, if preserved following resection. This region should be tolerant to depth electrode penetration if appropriately posterior to avoid Broca's area on the left hemisphere and appropriately anterior to avoid the pre-central gyms and associated corticospinal tract given the potential for adverse clinical outcomes.

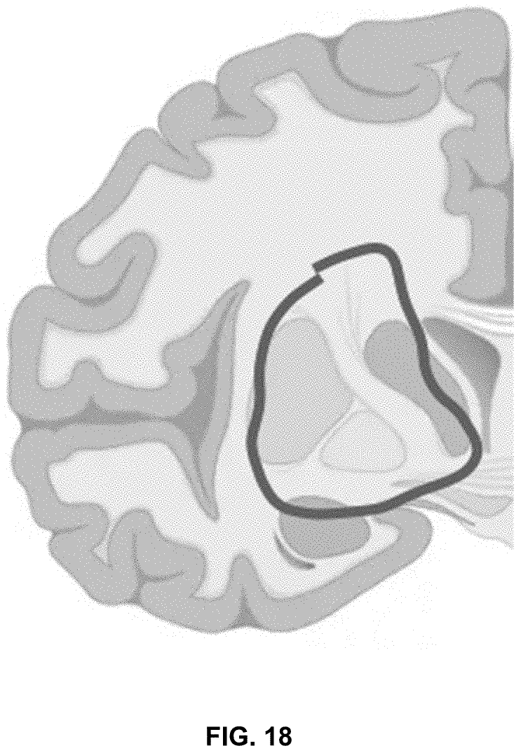

FIG. 18 is a demonstration of the “basal ganglia” sub-region of the cerebrum. This zone of cortical anatomy inclusive of the caudate, putamen, globus pallidus, internal capsule, external capsule, and other smaller volume subcortical tracts and is projected to be optimally treated using an implantation strategy involving depth electrodes given a subtotal region is this region is the most common clinical scenario, or rarely a resection cavity conformable grid with projecting electrodes when an aggressive resection is performed. The inter- and intra-contact distances along the grid and projecting/depth electrodes will be determined by the FEM simulation within the segmented model. Similarly, the spacing between the modular grid elements (described in FIG. 3) will be determined based on the FEM simulations. Depth electrode entry will be within the superior or middle frontal gyms, if preserved following resection. Depth electrode number and the spatial relationship will be determined based on FEM simulations. The putamen and caudate would likely be tolerant targets. Most likely intra-tumoral placement will be ideal given the frequency of subtotal resections (or no resection is likely for this lesion type). Although many sub-regions have been described separately herein, in other examples, tumors may be located across multiple sub-regions or even multiple regions. As such, after resection, depth electrode targets may occur within multiple sub-regions. In some examples, depth electrode trajectories may go through gyri and/or avoid the sulci (e.g., to reduce bleeding risk).

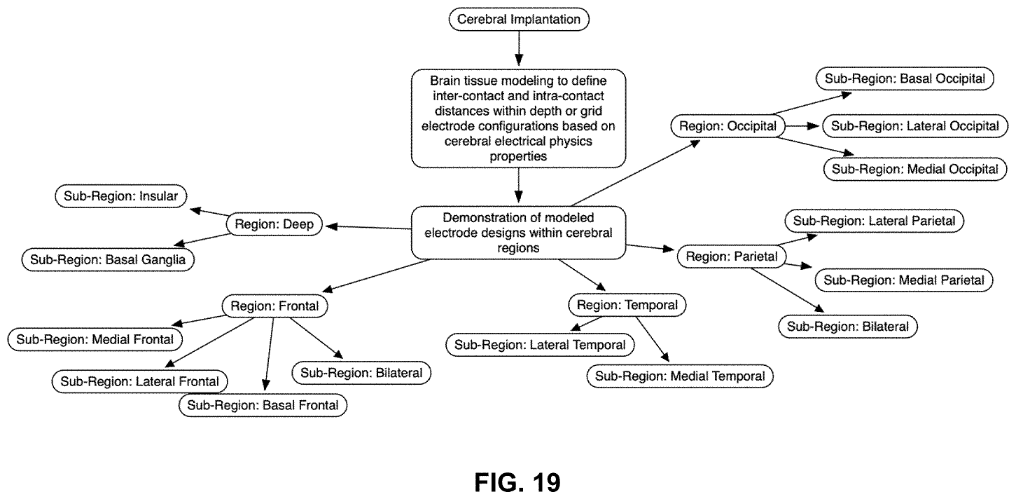

FIG. 19 is a flow chart illustrating the cerebral implantation strategy using electric field generation. This chart provides example decisions as to where electrodes may be implanted based on the brain tissue modeling and appropriate electrode depths and configurations.

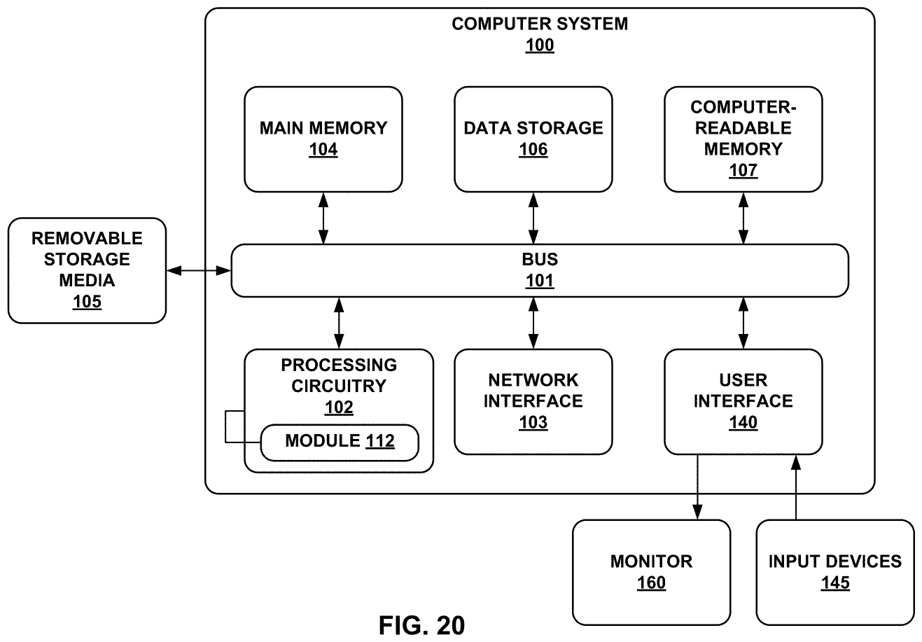

FIG. 20 illustrates an example of a computing system 100 configured to implement various aspects of the present system and methods for cerebral implantation strategy. Example embodiments described herein may be implemented at least in part in electronic circuitry; in computer hardware executing firmware and/or software instructions; and/or in combinations thereof. Example embodiments also may be implemented using a computer program product (e.g., a computer program tangibly or non-transitorily embodied in a machine-readable medium and including instructions for execution by, or to control the operation of, a data processing apparatus, such as, for example, one or more programmable processors or computers). A computer program may be written in any form of programming language, including compiled or interpreted languages, and may be deployed in any form, including as a stand-alone program or as a subroutine or other unit suitable for use in a computing environment. Also, a computer program can be deployed to be executed on one computer, or to be executed on multiple computers at one site or distributed across multiple sites and interconnected by a communication network.

Certain embodiments are described herein as including one or more modules 112. Such modules 112 are hardware-implemented, and thus include at least one tangible unit capable of performing certain operations and may be configured or arranged in a certain manner. For example, a hardware-implemented module 112 may comprise dedicated circuitry that is permanently configured (e.g., as a special-purpose processor, such as a field-programmable gate array (FPGA) or an application-specific integrated circuit (ASIC)) to perform certain operations. A hardware-implemented module 112 may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or processing circuitry may be configured by software (e.g., an application or application portion) as a hardware-implemented module 112 that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules 112 are temporarily configured (e.g., programmed), each of the hardware-implemented modules 112 need not be configured or instantiated at any one instance in time. For example, where the hardware-implemented modules 112 comprise a general-purpose processor configured using software, the general-purpose processor may be configured as respective different hardware-implemented modules 112 at different times. Software may accordingly configure a processing circuitry 102, for example, to constitute a particular hardware-implemented module at one instance of time and to constitute a different hardware-implemented module 112 at a different instance of time.

Hardware-implemented modules 112 may provide information to, and/or receive information from, other hardware-implemented modules 112. Accordingly, the described hardware-implemented modules 112 may be regarded as being communicatively coupled. Where multiple of such hardware-implemented modules 112 exist contemporaneously, communications may be achieved through signal transmission (e.g., over appropriate circuits and buses) that connect the hardware-implemented modules. In embodiments in which multiple hardware-implemented modules 112 are configured or instantiated at different times, communications between such hardware-implemented modules may be achieved, for example, through the storage and retrieval of information in memory structures to which the multiple hardware-implemented modules 112 have access. For example, one hardware-implemented module 112 may perform an operation, and may store the output of that operation in a memory device to which it is communicatively coupled. A further hardware-implemented module 112 may then, at a later time, access the memory device to retrieve and process the stored output. Hardware-implemented modules 112 may also initiate communications with input or output devices.

As illustrated, the computing system 100 may be a general purpose computing device, although it is contemplated that the computing system 100 may include other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the general purpose computing device may include various hardware components, such as a processing circuitry 102, a main memory 104 (e.g., a system memory), and a system bus 101 that couples various system components of the general purpose computing device to the processing circuitry 102. The system bus 101 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing system 100 may further include a variety of computer-readable media 107 that includes removable/non-removable media and volatile/nonvolatile media, but excludes transitory propagated signals. Computer-readable media 107 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the general purpose computing device. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 104 includes computer storage media in the form of volatile/nonvolatile memory such as read only memory (ROM) and random access memory (RAM). A basic input/output system (BIOS), containing the basic routines that help to transfer information between elements within the general purpose computing device (e.g., during start-up) is typically stored in ROM. RAM typically contains data and/or program modules that are immediately accessible to and/or presently being operated on by processing circuitry 102. For example, in one embodiment, data storage 106 holds an operating system, application programs, and other program modules and program data.

Data storage 106 may also include other removable/non-removable, volatile/nonvolatile computer storage media. For example, data storage 106 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules and other data for the general purpose computing device 100.

A user may enter commands and information through a user interface 140 or other input devices 145 such as a tablet, electronic digitizer, a microphone, keyboard, and/or pointing device, commonly referred to as mouse, trackball or touch pad. Other input devices 145 may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices 145 are often connected to the processing circuitry 102 through a user interface 140 that is coupled to the system bus 101, but may be connected by other interface and bus structures, such as a parallel port, game port or a universal serial bus (USB). A monitor 160 or other type of display device is also connected to the system bus 101 via user interface 140, such as a video interface. The monitor 160 may also be integrated with a touch-screen panel or the like.

The general purpose computing device may operate in a networked or cloud-computing environment using logical connections of a network interface 103 to one or more remote devices, such as a remote computer. The remote computer may be a personal computer, a server, a router, a network PC, a peer device or other common network node, and typically includes many or all of the elements described above relative to the general purpose computing device. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the general purpose computing device may be connected to a public and/or private network through the network interface 103. In such embodiments, a modem or other means for establishing communications over the network is connected to the system bus 101 via the network interface 103 or other appropriate mechanism. A wireless networking component including an interface and antenna may be coupled through a suitable device such as an access point or peer computer to a network. In a networked environment, program modules depicted relative to the general purpose computing device, or portions thereof, may be stored in the remote memory storage device.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
processing circuitry configured for operative communication with a conformable grid, a user interface, and a memory, wherein the conformable grid comprises a plurality of modular grid elements having a plurality of electrodes configured for implantation in a cerebrum of a patient, and wherein the processing circuitry is configured to execute instructions stored in the memory to:
model brain tissue to define inter-contact and intra-contact distances along the conformable grid and each of the plurality of electrodes;
determine the spacing between the plurality of modular grid elements of the conformable grid;
control the user interface to display a visual representation of the cerebrum including identification of a sub-region of the cerebrum; and
control the user interface to display a representation of the spacing between the plurality of modular grid elements of the conformable grid and a depth of each electrode of the plurality of electrodes in the cerebrum within the sub-region of the cerebrum.

2. The system of claim 1, wherein the processing circuitry is configured to define the inter-contact and intra-contact distances along the conformable grid by at least running a finite element model (FEM) stimulation within the model brain tissue.

3. The system of claim 1, wherein the processing circuitry is configured to generate the model brain tissue as a segmented cerebral model having a segmented 3D mesh.

4. The system of claim 3, wherein the processing circuitry is configured to manipulate the segmented 3D mesh to determine the spacing.

5. The system of claim 1, wherein the processing circuitry is configured to determine, based on the distances and spacing, a number of electrodes of the conformable grid for implantation in the cerebrum.

6. The system of claim 1, wherein the processing circuitry is configured to determine, based on the distances and spacing, a location of each electrode of the plurality of electrodes within the cerebrum.

7. The system of claim 1, further comprising the conformable grid comprising the plurality of modular grid elements having the plurality of electrodes.

8. The system of claim 1, further comprising the memory.

9. The system of claim 1, further comprising the user interface.

10. The system of claim 9, wherein the user interface comprises a display device configured to display the visual representation of the cerebrum including identification of a sub-region of the cerebrum and the representation of the spacing between the plurality of modular grid elements of the conformable grid and a depth of each electrode of the plurality of electrodes in the cerebrum within the sub-region of the cerebrum.

11. A method comprising:
modeling, by processing circuitry, brain tissue to define inter-contact and intra-contact distances along a conformable grid and each of a plurality of electrodes, wherein the comfortable grid comprises a plurality of modular grid elements having the plurality of electrodes configured for implantation in a cerebrum of a patient;
determining, by the processing circuitry, the spacing between the plurality of modular grid elements of the conformable grid;
controlling, by the processing circuitry, a user interface to display a visual representation of the cerebrum including identification of a sub-region of the cerebrum; and
controlling, by the processing circuitry, the user interface to display a representation of the spacing between the plurality of modular grid elements of the conformable grid and a depth of each electrode of the plurality of electrodes in the cerebrum within the sub-region of the cerebrum.

12. The method of claim 11, wherein defining the inter-contact and intra-contact distances along the conformable grid comprises running a finite element model (FEM) stimulation within the model brain tissue.

13. The method of claim 11, further comprising generating the model brain tissue as a segmented cerebral model having a segmented 3D mesh.

14. The method of claim 13, further comprising manipulating the segmented 3D mesh to determine the spacing.

15. The method of claim 11, further comprising determining, based on the distances and spacing, a number of electrodes of the conformable grid for implantation in the cerebrum.

16. The method of claim 11, wherein further comprising determining, based on the distances and spacing, a location of each electrode of the plurality of electrodes within the cerebrum.

17. The method of claim 11, wherein the processing circuitry is configured to be in operative communication with the conformable grid comprising the plurality of modular grid elements having the plurality of electrodes.

18. The method of claim 11, further comprising obtaining instructions from a memory, the instructions defining the modeling of the brain tissue.

19. The method of claim 11, further comprising displaying by a display device of the user interface, the visual representation of the cerebrum including identification of a sub-region of the cerebrum and the representation of the spacing between the plurality of modular grid elements of the conformable grid and a depth of each electrode of the plurality of electrodes in the cerebrum within the sub-region of the cerebrum.

20. A computer-readable storage medium comprising instructions that, when executed, cause processing circuitry to:
model brain tissue to define inter-contact and intra-contact distances along a conformable grid and each of a plurality of electrodes, wherein the conformable grid comprises a plurality of modular grid elements having the plurality of electrodes configured for implantation in a cerebrum of a patient;
determine the spacing between the plurality of modular grid elements of the conformable grid;
control a user interface to display a visual representation of the cerebrum including identification of a sub-region of the cerebrum; and
control the user interface to display a representation of the spacing between the plurality of modular grid elements of the conformable grid and a depth of each electrode of the plurality of electrodes in the cerebrum within the sub-region of the cerebrum.
